(19) United States
(12) Patent Application Publication
(10) Pub. No.: US 2024/0294549 A1
(43) Pub. Date: Sep. 5, 2024

(54) AN IMPROVED PROCESS FOR THE PREPARATION OF TRABECTEDIN

(71) Applicant: NATCO PHARMA LIMITED, Hyderabad (IN)

(72) Inventors: Sunitha KOTHAMUNIREDDYGARI, Hyderabad (IN); Anitha GATTU, Hyderabad (IN); Rajireddy SOLIPURAM, Hyderabad (IN); Srinivasa Chary CHINTALAPATI, Hyderabad (IN); Thirupathi KOTTE, Hyderabad (IN); Veerababu MADALAPU, Hyderabad (IN); Shankar Reddy BUDIDETI, Hyderabad (IN); Pulla Reddy MUDDASANI, Hyderabad (IN); Venkaiah Chowdary NANNAPANENI, Hyderabad (IN)

(21) Appl. No.: 18/686,927

(22) PCT Filed: Aug. 30, 2022
(86) PCT No.: PCT/IN2022/050769

§ 371 Date: Feb. 27, 2024

(30) Foreign Application Priority Data
Aug. 31, 2021 (IN) 202141039392

(51) Int. Cl. C07D 515/22

(52) U.S. Cl. CPC C07D 515/22

(57) ABSTRACT

The present invention is related to novel process for preparation of Trabectedin which involves less expensive reagents, solvents and the process conditions can be easily adopted for commercial scale.

### FIELD OF THE INVENTION

The present invention relates to an improved and industrially viable process for the preparation of Ecteinascidin derivatives i.e. Trabectedin. The present invention involves less expensive reagents, solvents and the process conditions can be easily adopted for commercial scale.

### BACKGROUND OF THE INVENTION

Trabectedin is Ecteinascidin Derivative. Trabectedin (Ecteinascidin 743 or ET-743) has a complex tris(tetrahydroisoquinolinephenol) structure has depicted below

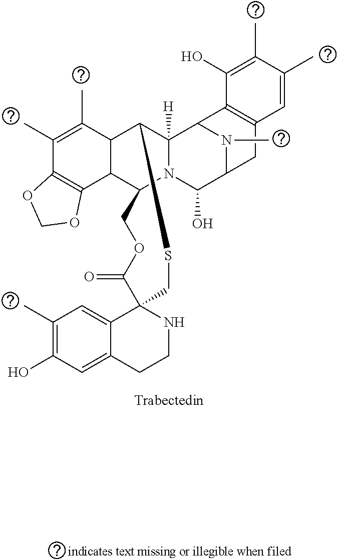

Trabectedin is approved under the brand name YONDELIS® and indicated for the treatment of patients with unresectable or metastatic liposarcoma or leiomyosarcoma

Trabectedin was originally prepared by isolation from extracts of the marine tunicate Ecteinascidia turbinata. The yield was low, and alternative preparative processes had been sought.

The first synthetic process for producing ecteinascidin compounds was described in U.S. Pat. No. 5,721,362. This process employed sesamol as starting material and yielded ET-743 after a long and complicated sequence of 38 examples each describing one or more steps in the synthetic sequence.

Angew Chem Int Ed Engl. 2019 Mar. 18; 58(12): 3972-3975 has reported process for the preparation of Trabectedin, wherein compound III was prepared from compound-IV by using 4-formyl-1-methylpyridiniumbenzenesulfonate, the reaction is not meeting large-scale requirement as it is giving low yield as well as inconsistent for completion of reaction. It also observed that the quality of 4-formyl-1-methylpyridiniumbenzenesulfonate is very important for the reaction success. Commercial availability of ultra-quality of 4-formyl-1-methylpyridiniumbenzenesulfonate is always is risk.

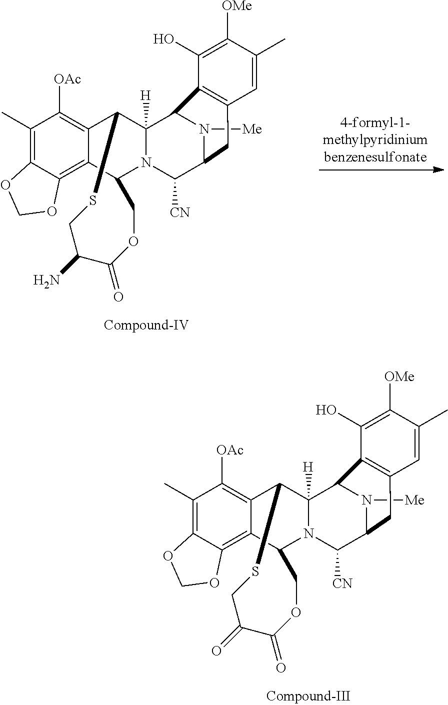

The inventors of the present invention have developed an alternative improved process for the preparation of Trabectedin. The present process is simple, cost effective and feasible in large scale production.

### OBJECT OF THE INVENTION

One object of the present invention is to provide a process for the preparation of Trabectedin, which is simple, economical, and suitable for industrial scale up.

### SUMMARY OF INVENTION

One aspect of the present invention of relates to process for the preparation of Trabectedin comprising the steps of:
a) reacting compound of formula-V with Zn under buffer conditions to get compound of formula-IV,
b) reacting compound of formula-IV with metal glyoxalate under buffer conditions to get compound of formula-III,
c) reacting compound of formula-III with 3-hydroxy-4-methoxyphenethyl amine in presence of organic solvent to obtain compound of formula-II,
d) reacting compound of formula-II with silver nitrate in presence of acetonitrile to get Trabectedin.

The process for the preparation of Trabectedin as per the present invention is depicted in the below scheme.

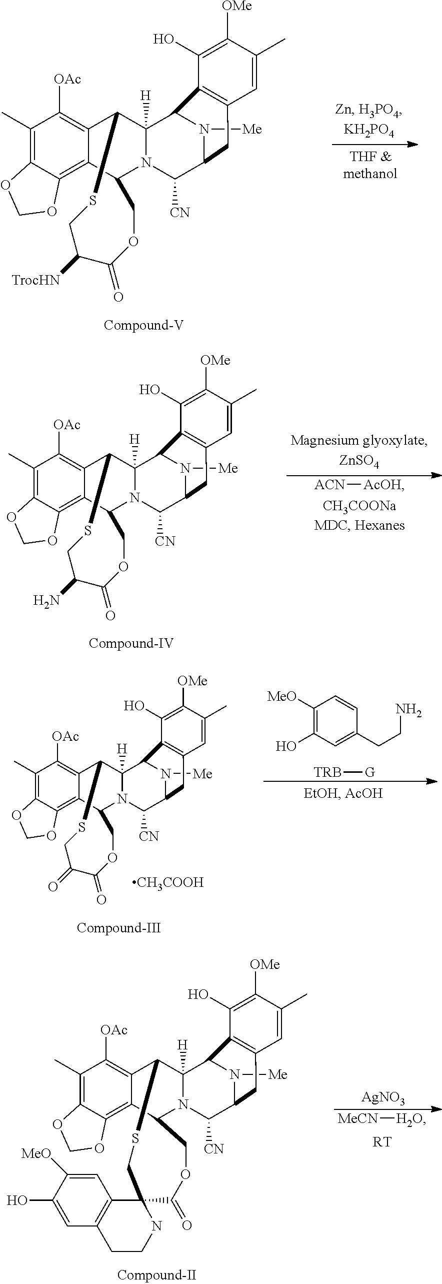

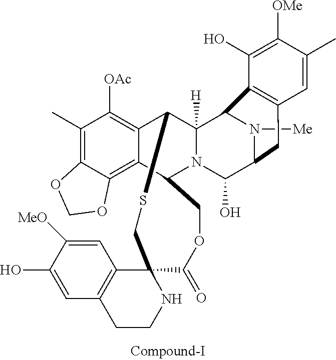

### DETAILED DESCRIPTION OF THE INVENTION

One embodiment of the present invention of relates to process for the preparation of Trabectedin comprising the steps of:
a) reacting compound of formula-V with Zn under buffer conditions to get compound of formula-IV.
b) reacting compound of formula-IV with metal glyoxalate under buffer conditions to get compound of formula-III,
c) reacting compound of formula-III with 3-hydroxy-4-methoxyphenethyl amine in presence of organic solvent to obtain compound of formula-II,
d) reacting compound of formula-II with silver nitrate in presence of acetonitrile to get Trabectedin.

As per the present invention preparation of Compound-IV was dissolved in organic solvent preferably acetonitrile and a freshly prepared buffer solution by using NaOAc and AcOH. In addition, above, anhydrous zinc sulfate and metal glyoxylate or mixture of glyoxylate or thereof preferably Magnesium glyoxylate. The reaction mass was stirred 25-30° C., the reaction completion was monitored and diluted with organic solvent preferably Dichloromethane; organic layer was washed with water. The organic layer was concentrated, and the crude compound was isolated by from hexane and in-situ intermediate Compound-III was prepared.

Compound-III was suspended in organic solvent such as methanol, ethanol, preferably ethanol and to that 3-hydroxy-4-methoxyphenethyl amine and organic acid preferably acetic acid was added in the reaction mass and reaction mass was maintained at room temperature followed by 25-30° C. for 4 hours and monitored the reaction by HPLC/TLC. Crude was purified by chromatography to afford Compound-II.

A solution of Compound-II was dissolved in aqueous acetonitrile and Silver nitrate was added portion wise at 20-25° C., further maintained the reaction. The progress of the reaction was monitored by HPLC/TLC. The reaction mass was extracted with Dichloromethane afforded crude Compound-I and further this compound Trabectedin is recrystallized form ethanol to get the pure Trabectedin.

In a process for the preparation of Trabectedin wherein the metal glyoxylate used is magnesium glyoxylate.

In a process for the preparation of Trabectedin, wherein organic solvent used in step c) is selected from methanol, ethanol, isopropanol preferably ethanol.

We surprisingly found that alternate of 4-formyl-1-methylpyridinium benzenesulfonate that is metal glyoxalate and it provides good yield.

### Advantages of the Present Invention

1. use of metal glyoxylate provides good yield.
2. The process of the present invention is feasible to produce on commercial scale of trabectedin without any apprehension.
3. The obtained trabectedin by employing the present invention is highly pure.

The present invention is further illustrated in detail with reference to following examples. It is desired that the examples be considered in all respect as illustrative and are not intended to limit the scope of the invention in any way.

### Experimental Procedure

### Preparation of Trabectedin

### Example-1: Preparation of Compound of Formula-IV

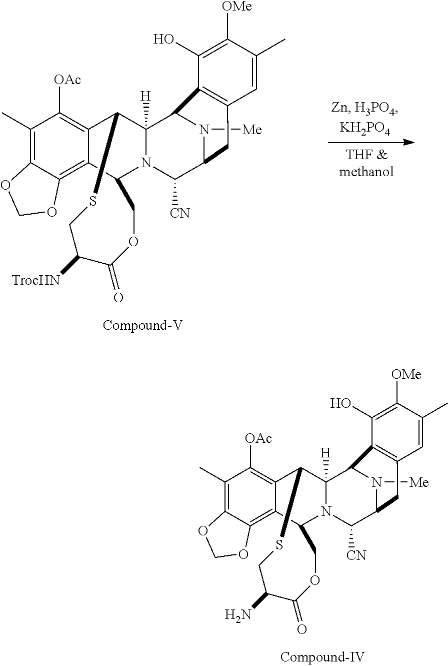

Compound-V (10.0 g) was dissolved in THF (350.0 ml), Methanol (125.0 ml) and a freshly prepared buffer solution (prepared by dissolving 7.19 g of H₃PO₄ and 10.85 g of KH₂PO₄ in 80.0 ml of water) was added lot-wise 65.6 g of Zn-dust to the reaction system at 25-30° C. The reaction mass was stirred for 40 min at 23-29° C., quenched with methylene chloride (2000 ml) and ˜8%, NaHCO₃ solution filtered and organic layer was washed with ˜15% sodium bicarbonate solution and brine solution. The organic layer was concentrated, purified by flash column chromatography using MDC and methanol solvents to yield compound-IV (5.9 g; 76.0%).

### Example-2: Preparation of Compound of Formula-III

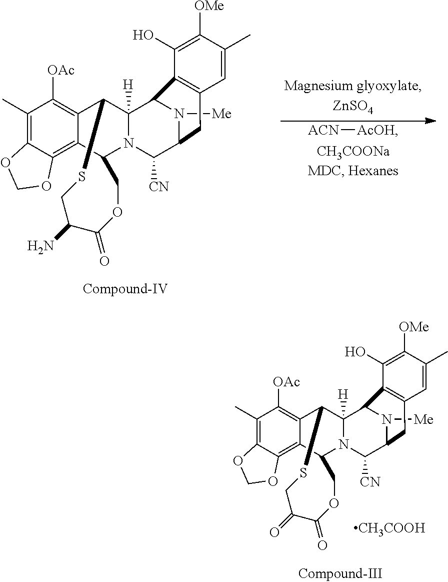

Compound-IV (9.1 g) was dissolved in acetonitrile (146.0 ml) and a freshly prepared buffer solution (prepared by dissolving 910.0 mg of NaOAc in 146.0 ml of AcOH) and anhydrous zinc sulfate (1.18 g) was added 14.9 g of magnesium glyoxylate to the reaction system at 25-30° C. The reaction mass was stirred for 60-75 min at 25-30° C., diluted with MDC (750.0 ml), organic layer was washed with water. The organic layer was concentrated, and the crude compound was isolated from hexane to yield Compound-III (8.4 g; 92.3%).

### Example-3: Preparation of Compound of Formula-II

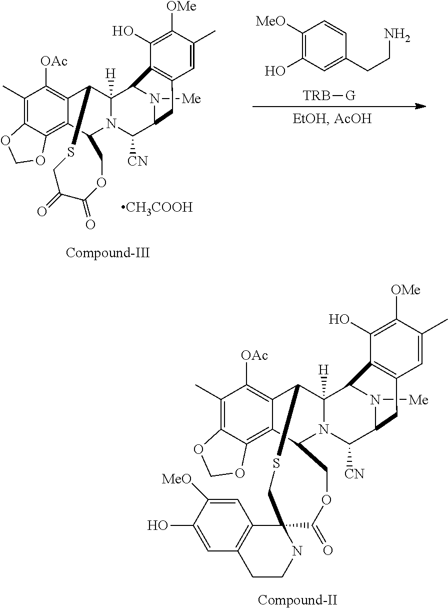

To a solution of compound-III (8.3 g), 3-hydroxy-4-methoxyphenethyl amine (3.05 g) and acetic acid (1.24 g) in anhydrous ethanol was added and stirred at 25-30° C. for 4.0 h. The reaction mixture was cooled to 0-5° C. and water (996.0 ml) is added to reaction mass. Solid is filtered and wet compound is dissolved in methylene chloride. The organic layer was concentrated and the residue was purified by flash column chromatography to yield compound-II (7.4 g; 78.8%).

### Example-4: Preparation of Trabectedin

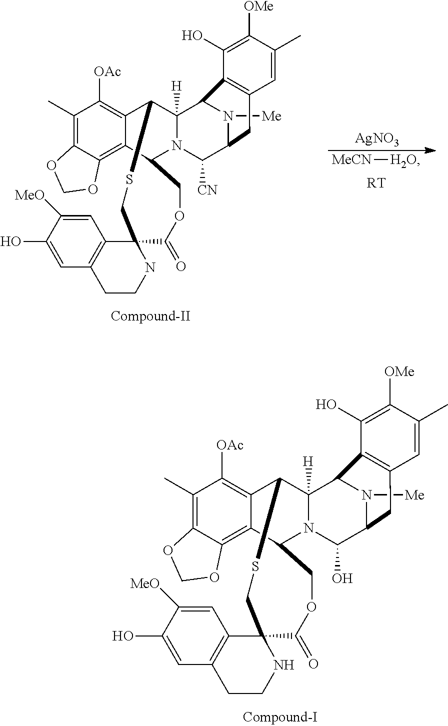

Compound-II (7.3 g) was dissolved in a mixture of ACN and water (3:2, 292 ml) and to this silver nitrate (16.1 g) was added in 2 lots. The reaction mass was stirred at 20-25° C. for 7.5 h, diluted with methylene chloride (250 ml), saturated sodium bicarbonate solution (125 ml) and brine solution (125 ml). The organic layer was separated, concentrated and the residue was purified by flash column chromatography to yield Trabectedin. (6.05 g; 82.8%).

### Example-5: Purification of Trabectedin

A solution of Trabectedin (2.1 g), in anhydrous ethanol (32.0 ml) was added to distilled water (960.0 ml) at 17-22° C. and stirred for 120 min at 17-22° C. The product was filtered, washed and dried to yield pure Trabectedin (1.84 g with 99.9% purity, deshydroxy impurity: 0.02%, desacetyl impurity: 0.02%, sulfoxide impurity: Nil).

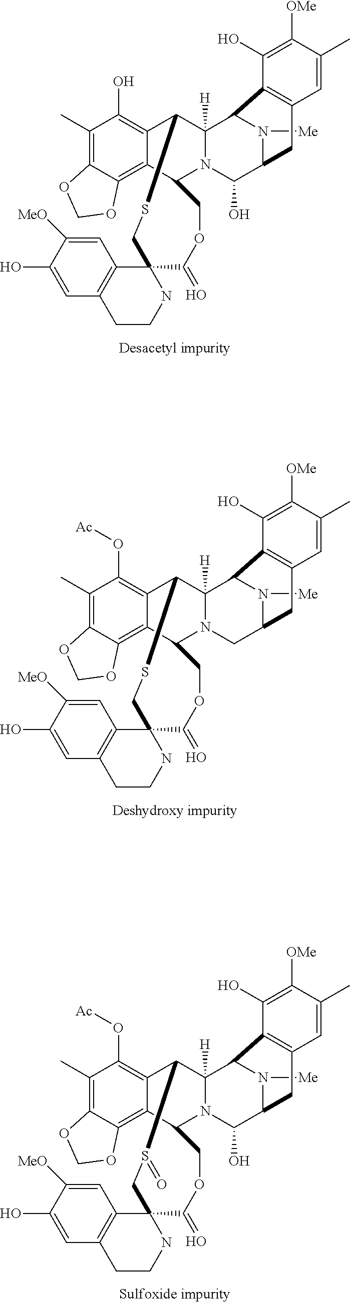

We claim:

1. A process for the preparation of Trabectedin comprising the steps of:
a) reacting compound of formula-V with Zn under buffer conditions to get compound of formula-IV,
b) reacting compound of formula-IV with metal glyoxalate under buffer conditions to get compound of formula-III,
c) reacting compound of formula-III with 3-hydroxy-4-methoxyphenethyl amine in presence of organic solvent to obtain compound of formula-II,
d) reacting compound of formula-II with silver nitrate in presence of acetonitrile to get Trabectedin.

2. A process as claimed in claim 1, wherein step a) the buffer solution is prepared by dissolving H₃PO₄ and KH₂PO₄ in water.

3. A process as claimed in claim 1, wherein step b) the buffer solution is prepared by dissolving NaOAc in AcOH.

4. A process as claimed in claim 1, wherein step b) the metal glyoxylate used is magnesium glyoxylate.

5. A process as claimed in claim 1, wherein step b) in addition to metal glyoxylate, Zinsulfate is also used in the reaction.

6. A process as claimed in claim 1, wherein organic solvent used in step c) is selected from methanol, ethanol, isopropanol.
